(19) United States
(12) Patent Application Publication
(10) Pub. No.: US 2019/0167198 A1
(43) Pub. Date: Jun. 6, 2019

(54) TREATMENT BED

(71) Applicants: CAPICAL GMBH, Braunschweig (DE); LIKAMED GMBH, Eppingen (DE)

(72) Inventors: Henning BÖGE, Braunschweig (DE); Martin OEHLER, Braunschweig (DE); Yvonne FRANK, Gemmingen (DE); Bernhard GAUPP, Eppingen (DE)

(73) Assignees: CAPICAL GMBH, Braunschweig (DE); LIKAMED GMBH, Eppingen (DE)

(21) Appl. No.: 16/313,715

(22) PCT Filed: Jun. 27, 2017
(86) PCT No.: PCT/EP2017/065856

§ 371 Date: Jan. 15, 2019

(30) Foreign Application Priority Data
Jul. 6, 2016 (DE) 10 2016 112 391.9

(51) Int. Cl. A61B 5/00; A61B 5/0408; A61B 5/024; A61B 5/0428

(52) U.S. Cl. CPC A61B 5/6891; A61B 5/04085; A61B 5/04284; A61B 5/02405; A61B 5/746

(57) ABSTRACT

The invention relates to a treatment bed for supporting patients in a sitting and/or lying manner for the duration of a treatment and/or diagnosis. The treatment bed has a support surface which consists of one or more segments and on which the patient is supported during the treatment and/or diagnosis. Multiple capacitive measuring electrodes for the contactless capacitive detection of EKG signals of a patient supported on the support surface are arranged in at least one segment of the support surface on the surface side closer to the patient. The treatment bed further has at least one electronic signal processing system which is connected to the measuring electrodes and is designed to process signals, in particular to amplify signals, of the electric signals of the measuring electrodes. In addition to the measuring electrodes, the treatment bed also has at least one injection electrode which is designed to teed injection signals into one or more of the measuring electrodes via the patient supported on the support surface. The electronic signal processing system is additionally designed to determine the quality of the capacitive coupling of one or more or all of the measuring electrodes to the patient by means of the signals received via the measuring electrodes using the signal components which are contained in the signals and originate from the injection signals.

The invention relates to a treatment couch for supporting patients in a sitting and/or lying position for the duration of a treatment and/or diagnosis. Such treatment couches are known for example from the product range of Likamed GmbH.

The invention is based on the object of further developing such a treatment couch with regard to its functionality and making it more universal.

This object is achieved according to claim 1 by a treatment couch for supporting patients in a sitting and/or lying position for the duration of a treatment and/or diagnosis, wherein the treatment couch has a supporting surface which consists of one or more segments and on which the patient is supported during the treatment and/or diagnosis, wherein multiple capacitive measuring electrodes for the contactless capacitive detection of ECG signals of a patient supported on the supporting surface are arranged in at least one segment of the supporting surface, on the side of the surface thereof that is near the patient, wherein the treatment couch also has at least one electronic signal processing system, which is connected to the measuring electrodes and is designed for signal processing, in particular for signal amplification, of the electrical signals of the measuring electrodes, wherein the treatment couch has in addition to the measuring electrodes at least one injection electrode, which is designed for feeding injection signals into one or more of the measuring electrodes via the patient supported on the supporting surface, wherein the electronic signal processing system is also designed to determine from the signals received by way of the measuring electrodes, on the basis of the signal components that are contained therein and originate from the injection signals, the quality of the capacitive coupling of one or more or all of the measuring electrodes to the patient. The segments of the supporting surface may for example comprise a back, foot and/or seat segment. The treatment may in particular be a medical treatment, for example a treatment with pharmaceutical applications.

The treatment couch according to the invention has the advantage that biosignals, in particular ECG signals (ECG=electrocardiogram) can be recorded in an easy way from a patient resting on the treatment couch. For this, it is not necessary for the patient to be rather laboriously connected to electrodes, as in the case of conventional ECG devices. Instead, incorporated at suitable points in the treatment couch are capacitive measuring electrodes, with which such ECG signals can be contactlessly recorded.

Advantageously, the treatment couch also has an electronic signal processing system, by which the signals of the measuring electrodes can be processed, wherein in particular a signal amplification can take place. In this way, the treatment couch can provide ECG signals of high quality. The electronic signal processing system may take the form of an individual electronic system or the form of multiple electronic components. In the case of multiple electronic components, in particular a local electronic signal-amplification component may be arranged in the vicinity of a respective measuring electrode.

Also incorporated in the treatment couch apart from the measuring electrodes is also at least one injection electrode. In an advantageous embodiment of the invention, it is provided that the treatment couch has in addition to the measuring electrodes at least two injection electrodes, which are electrically separate from one another and are designed for feeding injection signals into one or more of the measuring electrodes via the patient supported on the supporting surface. By means of the injection electrode or the injection electrodes, an automatic check on the quality of the capacitive coupling of the measuring electrodes to the patient can be performed by feeding in injection signals. In this way, when the ECG signals do not occur or deteriorate, it can be automatically distinguished whether the ECG signals have changed because of a change in position of the patient, and accordingly deteriorated capacitive coupling, or because of a deterioration in the health of the patient. This health of the patient is detected. In this way, the safety of the patient can be improved, in particular in the case of patients undergoing onerous treatments. What is more, improved control of the treatment is possible. For instance, the alarm signal can be generated at an early time, so that medical personnel can initiate countermeasures at an early time.

In particular, the injection electrodes are not designed for detecting ECG signals. Nevertheless, the injection electrodes may be structurally formed in a way comparable to the measuring electrodes, for example as capacitive injection electrodes. The injection electrodes may also be differently formed, for example as galvanic injection electrodes, which must be brought into galvanic contact with the skin of the patient.

If the measuring electrodes and/or the injection electrodes are formed as capacitive electrodes, it is advantageous to arrange them in the treatment couch in the covering material of the supporting surface or in the covering material of pads of the supporting surface, or under the covering material. In this way, the measuring electrodes and/or the injection electrodes can be integrated in the treatment couch, so that they are not visible from the outside. In this way, the visual appearance of the treatment couch is not changed by the integration of the measuring electrodes and/or injection electrodes. It is also possible to attach the measuring electrodes and/or the injection electrodes visibly on the supporting surface.

According to an advantageous development of the invention, it is provided that one or more or all of the measuring electrodes and/or the first and/or the second injection electrode is/are formed as textile capacitive electrodes, which are embedded in a structure near the surface of the side of the supporting surface near the patient. In this way, the measuring electrodes and/or the injection electrodes can be integrated into the material of the treatment couch in a particularly favorable way. This makes it structure, which do not disturb a patient supported on the treatment couch and also do not leave behind any damage, such as pressure points or the like.

According to an advantageous development of the invention, it is provided that the electronic signal processing system is arranged on the treatment couch away from the measuring electrodes and/or the injection electrodes. This is also advantageous for the comfortable support of the patient. Since the electronic signal processing system, in particular the electronic signal-amplification components to be arranged in the vicinity of the measuring electrodes, generally consist of material that is hard, or at least less flexible than the electrodes, this development of the invention can have the effect of avoiding an adverse effect on the patient supported on the treatment couch.

The ECG signals recorded by way of the measuring electrodes may be displayed by the electronic signal processing system, for example on a display device of the treatment couch, and/or be stored in a memory of the treatment couch for purposes of documentation.

According to a further development of the invention, it is provided that the treatment couch has an electrical terminal connector, by way of which a treatment monitor can be electrically coupled to the treatment couch and its electronic signal processing system, wherein the electronic signal processing system is designed to emit on the basis of the signals of the measuring electrodes ECG signals of the patient in a normalized form by way of the terminal connector. This has the advantage that a commercially available treatment monitor can be used for the display and documentation of the detected ECG signals. Accordingly, the treatment couch, including with the enhancements according to the invention, can remain manageable in terms of the technical complexity, and accordingly be provided at low cost.

The treatment couch may in particular have one or more of the following further features:
A subframe produced from steel (powder-coated) together with up to four fixable rollers to provide stability, for supporting patients over several hours and for transporting patients over a relatively short distance.
A padded supporting surface produced from steel (powder-coated) for patients, consisting of at least one, itself-adjustable, to five individually adjustable elements:
   A complete supporting surface in one piece
   Back and foot parts individually adjustable, infinitely variably
   Back and foot parts and height individually adjustable, infinitely variably
   Back, foot and seat parts individually adjustable, infinitely variably
   Back, foot and seat parts and height individually adjustable, infinitely variably
   Back, foot and seat parts and foot support and height individually adjustable, infinitely variably
   The adjusting ranges of the supporting surface are equipped by one or up to five 24 V actuators (all with limit switches). All of the actuators can be moved individually by way of manual operation and/or foot switches. Depending on the configuration/equipment of the product, a sitting position, a bed position, a recovery position and/or a Trendelenburg position can be achieved.
Two arm rests attached to the produced/said steel structure (powder-coated) for supporting the arms/elbows
A padded support/mattress (various inner rests, covered with PU coating or synthetic leather).

As mentioned, the treatment couch may be designed for treatment with pharmaceutical applications. Accordingly, in one embodiment of the invention the treatment couch may not be designed for radiation therapies. It is suitable for example for supporting patients in cases of dialysis, blood donation, treatment of pain, therapies in the area of oncology and similar types of treatment. Accordingly, the treatment couch is intended for use in medical environments. Alternatively, the treatment couch may also be used in the home. For this purpose, the treatment couch may for example have an electrical terminal for additional equipotential bonding.

According to an advantageous development of the invention, it is provided that at least one segment is adjustable arbitrarily into different positions by at least one electric motor of the treatment couch. Thus, the treatment couch may have multiple electric motors for the adjustment of different segments of the supporting surface. The treatment couch may in this way be adjusted infinitely variably in a motorized manner, for example from the sitting or lying position into the recovery position or possibly into the Trendelenburg position. Therefore, according to an advantageous development of the invention, it is provided that the treatment couch is adjustable from the sitting position into a lying position and vice versa by at least one electric motor of the treatment couch.

According to an advantageous development of the invention, it is provided that the treatment couch is supported with respect to the floor on multiple fixable rollers. In this way, the treatment couch can be easily pushed from one position to another position, possibly also with a patient supported on it. At a desired position, the treatment couch can then be fixed by way of fixable rollers, so that it cannot readily roll away.

According to an advantageous development of the invention, it is provided that the treatment couch has to the left and right of the supporting surface at least one arm rest. Apart from increased comfort for the patient, this is particularly advantageous for certain types of treatment, for example for infusions.

According to an advantageous development of the invention, it is provided that the treatment couch has at least one acoustic and/or optical signal transmitter, wherein the electronic signal processing system is designed to activate the signal transmitter to issue an alarm signal when there are predetermined signal combinations of the detected ECG signals and the quality of the capacitive coupling.

Accordingly, in the case of certain signal combinations that indicate a deterioration in the state of health of the patient, the medical personnel can be informed in good time and automatically.

According to an advantageous development of the invention, it is provided that the injection electrodes comprise a first injection electrode and a second injection electrode, the injection signals comprise a first injection signal and a second injection signal, different from the first injection signal, the first injection signal is fed into the first injection electrode from the electronic signal processing system and, overlapping in time or at the same time, the second injection signal is fed into the second injection electrode from the electronic signal processing system.

By introducing additional injection electrodes, which are present in addition to the measuring electrode or the measuring electrodes of the system and accordingly do not serve as measuring electrodes, i.e. not for detecting electrical biosignals, it is possible to provide fixed feed-in paths for injection signals, which in a way corresponding to their fixed function only have to be provided with the electronic wiring required for this. As known from ECG systems, the injection electrodes may in particular be formed as ground electrodes in the sense of DRL electrodes (DRL—driven right leg). In this way, the injection electrodes may be formed as electrodes which, as a difference from the measuring electrodes, are already actively driven by respective amplifiers. Accordingly, the additional effort required for feeding in the injection signals is relatively low. Thus, for example, by means of an analog adder, an injection signal in the form of a sinusoidal signal can be added with little effort to the common-mode rejection signal that is in any case supplied in the case of a DRL electrode, or at least with much less effort than would be necessary in the case of a modification of measuring electrodes.

The system according to the invention has the advantage that the two injection electrodes that are separate from one another allow the feeding in of two different injection signals, which via the biosignal source can in turn be detected by means of the measuring electrode or the measuring electrodes and are distinguishable from one another. Accordingly, it is possible to determine from the signals received by way of the measuring electrode, on the basis of the signal components that are contained therein and originate from the first and second injection signals, the quality of the capacitive coupling of the measuring electrode with the biosignal source. Advantageously, it is always only the at least two injection electrodes with the injection signals that are different from one another that are required, irrespective of the number of measuring electrodes of the system used. Consequently, the detected signals can be used to determine for each measuring electrode the quality of its capacitive coupling to the biosignal source. For this purpose, the electronic signal processing system may for example have a filter for filtering out the signal components originating from the first and second injection signals.

As the result of determining the quality of the capacitive coupling of the measuring electrode to the biosignal source, a numerical value, for example a numerical value that reflects the coupling capacity, may for example be determined, or else, after corresponding pre-evaluation, a good/bad item of information, which indicates whether or not the electrical biosignals of the biosignal source that are detected by way of a measuring electrode can be meaningfully evaluated. In particular, the variation over time of the quality of the capacitive coupling determined in this way can be evaluated.

According to an advantageous development of the invention, only the first injection signal is fed into one injection electrode and an overlay of the first injection signal and the second injection signal is fed into the other injection electrode. According to an advantageous development of the invention, a common-mode rejection signal is additionally fed into both injection electrodes.

According to an advantageous development of the invention, the electronic signal processing system is designed for determining the quality of the capacitive coupling on the basis of the amplitude values and the phase positions of the signal components of the first and second injection signals that are received by way of the measuring electrode.

According to an advantageous development of the invention, the system is designed for determining the heart rate or a variable derived therefrom of the biosignal source.

According to an advantageous development of the invention, measured values of the currents fed in by way of the first and second injection electrodes by means of the supplied injection signals are supplied to the electronic signal processing system as further input variables and the electronic signal processing system is designed for determining the quality of the capacitive coupling while taking into account the supplied measured values of the currents fed into the first and second injection electrodes by means of the supplied injection signals. The currents fed in may for example be ascertained by measuring resistors (shunts). In this way, further electrical measured variables for the evaluation of the variables fed in by means of the injection electrodes can be detected, so that the computational determination of the quality of the capacitive coupling of the measuring electrode is simplified further.

A treatment couch in the context of the present invention may in particular be formed an open treatment couch, without a closed or largely closed chamber surrounding the treatment couch. In this way, the treatment couch and a patient supported on it are accessible from all sides. The treatment couch may have separate individual resting surfaces that are adjustable in relation to one another for the back rest, sitting surface and leg supporting surface (or leg supporting surfaces), so that the treatment couch can be adapted for the purpose of treatment in many respects to the personal needs and the physical stature of the patient. The treatment couch may furthermore have arm rests, which are arranged laterally to the left and right of the sitting surface and/or back rest. The treatment couch allows in particular treatment of a patient in an at least partially sitting position.

The invention is explained in more detail below on the basis of exemplary embodiments with the use of drawings, in which:

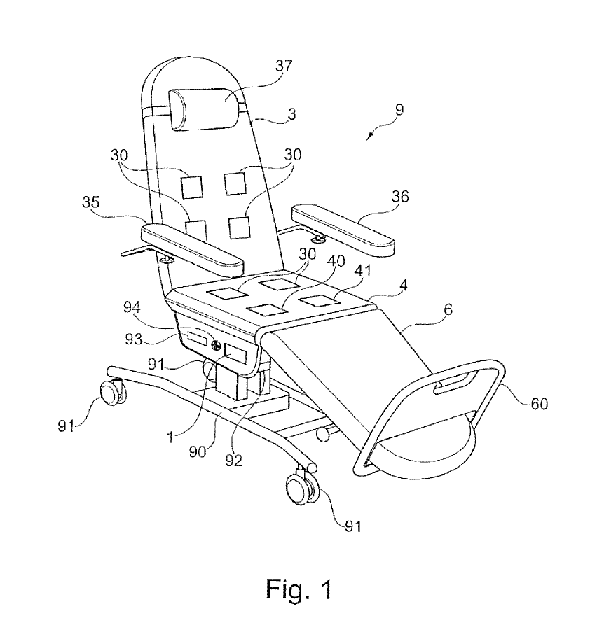

FIG. 1 shows a treatment couch in a perspective representation and

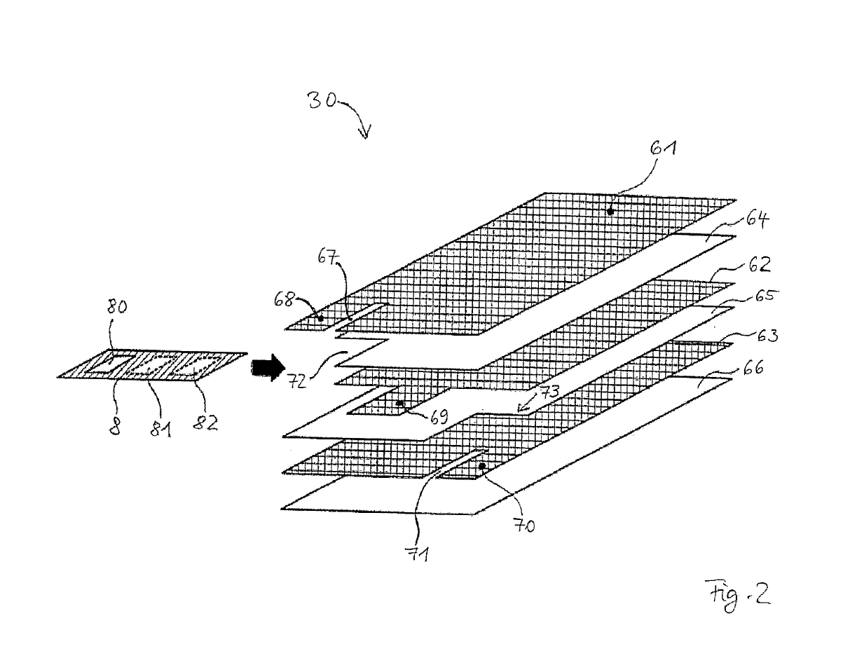

FIG. 2 shows the multi-layered structure of a textile electrode and

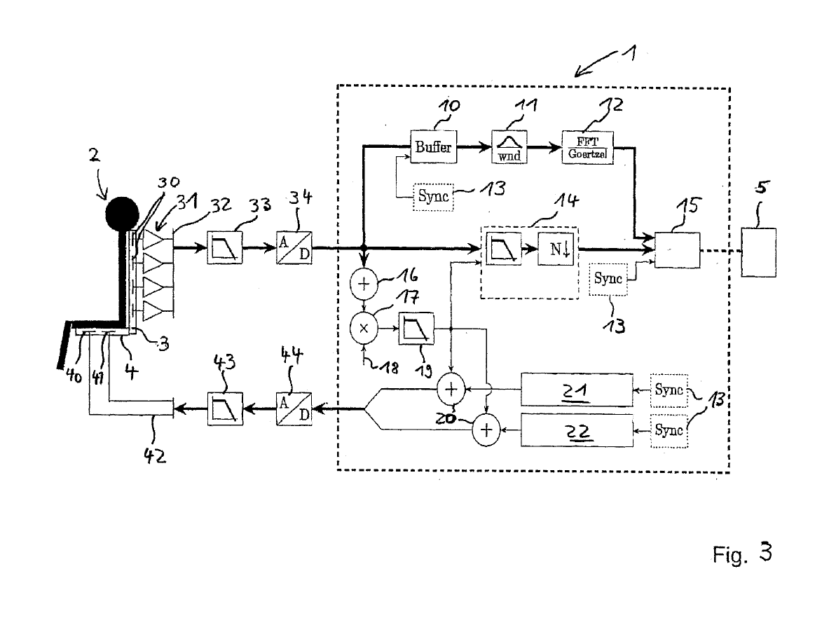

FIG. 3 shows the treatment couch according to FIG. 1 with regard to the electrical signal flows and

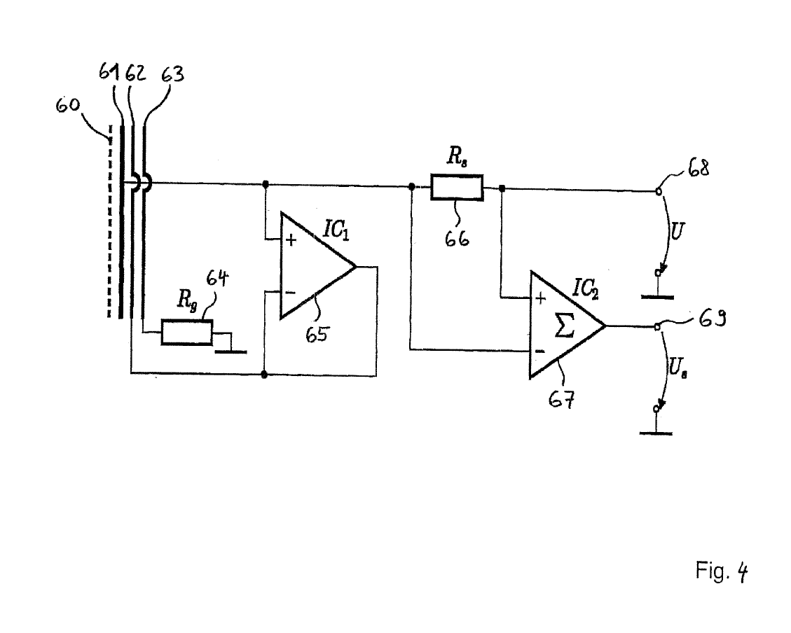

FIG. 4 shows a signal detecting circuit of a measuring electrode and

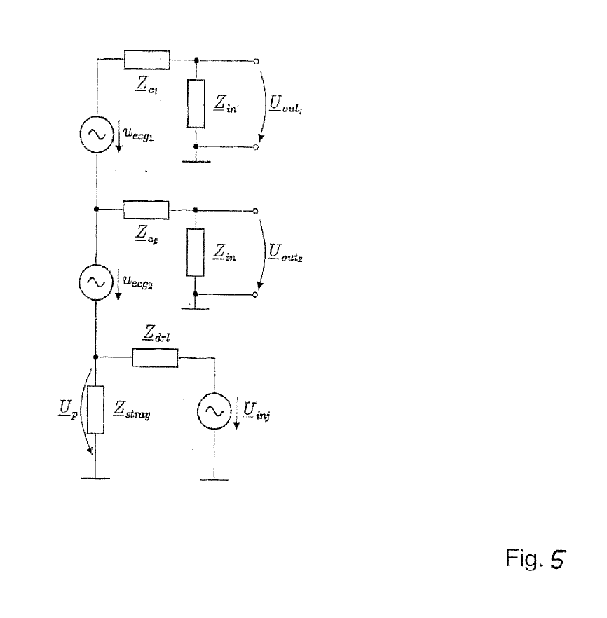

FIG. 5 shows an equivalent circuit diagram of a 2-channel system and

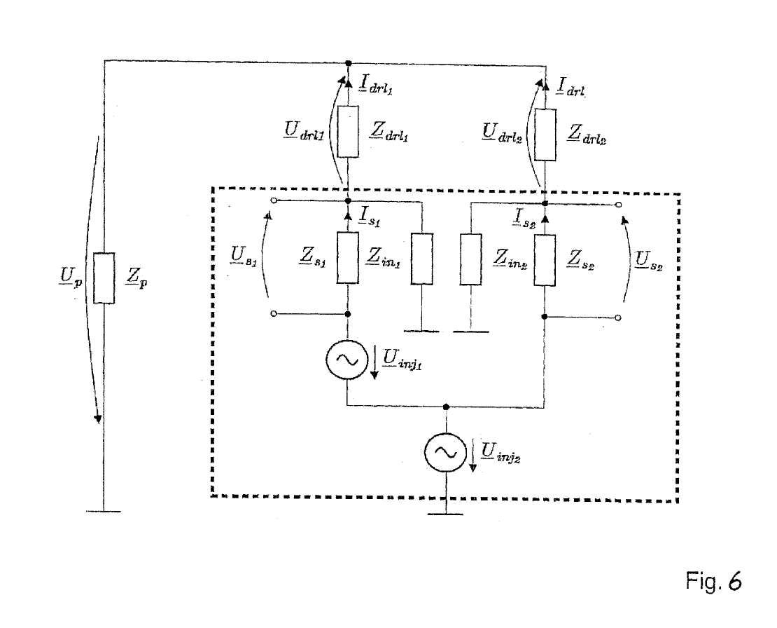

FIG. 6 shows an equivalent circuit diagram of the injection electrodes

In the figures, the same designations are used for elements that correspond to one another.

The treatment couch 9 shown in FIG. 1 has a supporting surface 3, 4, 6, which in this case is divided into three segments. The supporting surface has a back segment 3, serving as a back rest, a seat segment 4 and a foot segment 6, serving as a foot rest. The segments 3, 4, 6 of the supporting surface are in each case padded, wherein the padding material is covered with a covering material Integrated in the covering material or directly under the covering material are multiple electrodes 30, 30, 41. These electrodes 30, 40, 41 are not actually visible from the outside, but in FIG. 1 are depicted as visible elements to illustrate the invention. The electrodes 30, 40, 41 take the form of six capacitive measuring electrodes 30 and also a first capacitive injection electrode 40 and a second capacitive injection electrode 41. It is possible for example, as shown in FIG. 1, for four measuring electrodes 30 to be arranged in the back segment 3 and two measuring electrodes 30 to be arranged in the seat segment 4. Moreover, the first and second injection electrodes 40, 41 are likewise arranged in the seat element 4.

The treatment couch 9 has a subframe 90, which bears the supporting surface 3, 4, 6. The subframe 90 is supported on the floor by way of four rollers 91, which are fixable. By way of electric motors 92 arranged on the subframe 90 or in the vicinity of the segments 3, 4, 6, at least some of the segments, for example the back segment 3 and foot segment 6, can be adjusted electromotively into various positions.

The measuring electrodes 30 and the injection electrodes 40, 41 are electrically connected to an electronic signal processing system 1, arranged for example in the subframe 90. The electronic signal processing system 1 detects the signals of the capacitive measuring electrodes 30 and, to detect the quality of the capacitive coupling of the measuring electrodes 30 to the patient, also injects injection signals by way of the injection electrodes 40, 41 into the patient. The electronic signal processing system 1 may also be designed to process the recorded ECG signals in a normalized form and to emit them to the outside by way of a terminal connector 93, for example in the form of an electrical plug-in connector. Accordingly, a treatment monitor may be coupled to the terminal connector 93, in order to visually present the emitted normalized ECG signals and possibly document them.

The electronic signal processing system 1 may also be designed for monitoring the ECG signals in combination with the quality of the capacitive coupling of the measuring electrodes 30 to the patient for critical signal combinations. When a critical signal combination is detected, the electronic signal processing system 1 may activate a signal transmitter 94, in order to draw attention to the critical state.

The treatment couch 9 may furthermore have a left arm rest 36 and a right arm rest 35, also a head-rest element, arranged on the back segment 3, and a foot-resting surface 60, arranged on the foot segment 6.

FIG. 2 shows by way of example a textile electrode 1, as can be used as a measuring electrode 30 or else an injection electrode 40, 41.

FIG. 2 shows the textile electrode 1 with the individual layers in an isometric view before the layers are adhesively bonded together. Three electrically conductive layers 61, 62, 63 of an electrically conductive textile material and three insulating layers 64, 65, 66 of an insulating textile material can be seen. The uppermost electrically conductive layer 61 is the sensor layer of the electrode, which serves for the capacitive incoupling of the signal to be measured by means of the electrode. The middle electrically conductive layer 62 is a guard layer, which serves for shielding the sensor layer 61 from external interfering influences, in particular ESD influences. The lower electrically conductive layer 63 is a reference potential layer, which is to be connected to a reference potential. The sensor layer 61 has at a corner a clearance 67, through which a contact link 68 for the electrical contacting of the sensor layer 61 is formed. The guard layer 62 has a contact link 69, which is formed by pieces of textile material of the guard layer 62 to the left and right of the contact link 69 having been cut away. The contact link 69 serves for the electrical contacting of the guard layer 62. The reference potential layer 63 is formed in a way comparable to the sensor layer 61, but with a contact link 70 on the opposite side. The contact link 70 is formed as the result of a clearance 71, which is cut out from the textile material of the reference potential layer 63. The uppermost insulating layer 64 has at a corner a clearance 72, which lies underneath the contact link 68. The middle insulating layer 65 has at an opposite corner of the same side a clearance 73. The clearance 73 overlaps with the contact link 70. The lowermost insulating layer 66 does not have such clearances. The layers 61-66 may be brought into the outer contour described and shown for example by laser cutting.

The outer form of the electrode 1 or the individual layers 61-66 does not necessarily have to be substantially rectangular, as represented in FIG. 2, but may assume any other desired form, such as for example oval, rectangular with rounded corners or circular.

An electronic signal-amplification component 83, which serves for amplifying the electrical signals emitted by the capacitive textile electrode 1, is arranged in the vicinity of the textile electrode 1 shown in FIG. 2.

In this way, the treatment couch 9 with the technical elements explained represents a system for the capacitive detection of electrical biosignals from a biosignal source 2, i.e. from a patient. The function of such a system is explained in more detail below on the basis of FIGS. 3 to 6.

The system shown in FIG. 3 serves for the capacitive detection of electrical biosignals from a biosignal source 2, for example a human. For this, the treatment couch 9 is fitted with the corresponding capacitive measuring electrodes 30 and injection electrodes 40, 41. The injection electrodes 40, 41 are connected by way of separate electrical lines to devices 43, 44, which in FIG. 1 are only shown singly, but are provided separately for each injection electrode. The device 43 is a lowpass filter, for example with a cut-off frequency of 4 kHz. The device 44 is formed as a digital/analog converter, which converts a digital signal supplied by a central unit of the electronic signal processing system 1 into an analog voltage value and outputs it via the lowpass filter 43 to the respective injection electrode 40, 41.

The measuring electrodes 30 are connected by way of respective signal amplifiers 31, which may also be integrated in the respective textile electrode, to further signal processing means 33, 34. The measuring electrodes 30 or their signal amplifiers 31 may be connected in each case via an individual, separate signal path by way of signal processing means 33, 34 to the electronic signal processing system 1 or, if the complexity of the circuitry is to be reduced, be switched by way of a multiplexer 32 in each case to the same signal processing means 33, 34. The signal processing means 33 may be formed as a lowpass filter, for example with a cut-off frequency of 4 kHz. The signal processing means 34 may be formed as an analog/digital converter.

The respective analog/digital conversion or digital/analog conversion allows the signal processing to be performed completely digitally in the electronic signal processing system 1, with the advantage that signal processing algorithms of a relatively favorable complexity can be provided.

The electronic signal processing system 1 connected to the analog/digital converter 34 or the digital/analog converters 44 has the following structure. The digitized signals of the measuring electrode 30 that are detected by way of the analog/digital converter 34 are supplied to three different evaluation paths in the electronic signal processing system 1, to be precise one path for the evaluation of the signal components originating from the injection signals, one path for the ascertainment of the actual useful signals, to be specific the biosignals of the biosignal source, and one path that serves for common-mode rejection. First, the path for the evaluation of the signal components originating from the injection signals will be discussed. For this, first there is a buffer 10, in that the incoming data are first buffered in blocks, for example with a block size of 728 measured values. The block size is in this case chosen in particular such that full periods of the first and second injection signals are respectively stored in one block.

In a block 11, the signal components are filtered by a bandpass filter, for example by a non-rectangular window function, for example a Hanning filter. In a subsequent digital filter 12, a further filtering is performed, for example by means of a Fast Fourier Transform (FFT) or a Goertzel algorithm. The Goertzel algorithm allows the efficient determination of selected frequency components. With the data determined in this way, the quality of the capacitive coupling of the measuring electrode to the biosignal source, for example in the form of the coupling capacitance, can be determined in a block 15. The results of the quality determination can be output for example on a display device, for example a screen 5, or passed on for further processing.

By way of the filter block 14 shown approximately in the middle of the electronic signal processing system 1 in FIG. 1, the ECG signals are filtered out from the supplied signals of the measuring electrode. This may be performed for example by a two-stage FIR filter.

For the common-mode rejection, it is envisaged first to summate the supplied, digitized measuring signal by way of a summator 16. In this way, the common-mode signal is obtained. In a multiplier 17, the previously determined common-mode signal can also be amplified by a gain factor 18, for example in the range from 0 to 40 dB. The signal thereby formed is subsequently supplied to a further filter 19. The signal generated from the filter 19 is supplied on the one hand to the filter block 14, on the other hand to two summators 20.

In the blocks shown at the bottom in the electronic signal processing system 1, the first and second injection signals are generated in two signal generators 21, 22. The first injection signal may for example have a frequency of 1120 Hz at an amplitude of 100 mV, the second injection signal a frequency of 1040 Hz at an amplitude of 12.5 mV. Thus, the first signal generator 21 may be formed so as to directly emit an overlay of the first and second injection signals, while the other signal generator 22 only emits the first injection signal. In the summators 20, the signal emitted by the filter 19 is mixed with the respective injection signals to provide the common-mode rejection. The corresponding signals, which until then have been in a digital form, are converted by way of the already mentioned digital/analog converter 44 into analog signals and fed separately from one another via the filters 43 into the injection electrodes 40, 41.

For the dimensioning of the injection signals, a compromise has been found, allowing the injection signals to be placed at frequencies that are as close together as possible and offer a good demodulation rate, and at the same time allowing a sampling rates achievable for suitable precision analog/digital converters and available microcontrollers. Furthermore, the injection frequencies must be high enough to allow them to be sufficiently suppressed with respect to the useful signal (the ECG signal) by a single lowpass filter. As a result of this, a delimitation from movement artifacts, which lie in the range below 20 Hz, is also possible.

The amplitude of the injection signals also represents a compromise between a good signal-to-noise ratio and the lowest possible order of the lowpass filters, to allow simple signal processing.

FIG. 4 shows on the left by way of example a measuring electrode with the previously described multi-layered structure, which is arranged behind the textile surface 60 of the back segment 3. The reference potential layer 63 is connected to the system ground by way of a resistor 64. The sensor layer 61 is connected by way of a line first via an amplifier, for example an operational amplifier 65, to the guard layer 62. Furthermore, for detecting the already explained signals to be detected by way of the measuring electrode, the sensor layer 61 is connected to a measuring terminal 68, at which there is the measuring signal that is to be detected by means of the signal evaluation means 33, 34. To be able in addition to carry out a current measurement with respect to the injection electrodes 40, 41, a measuring resistor in the form of a shunt 66 is present on the line from the sensor layer 61 to the measuring terminal 68. The voltage dropping across the shunt 66, which is an indicator of the current flowing through it, is amplified by way of an amplifier 67 and delivered to an output terminal 69. The signal available at the output terminal 69 is supplied, possibly after prior filtering, likewise by way of analog/digital converters to the electronic signal processing system 1 and is further processed there.

The determination of the quality of the capacitive coupling, for example in the form of a coupling impedance, can be performed as follows. This is based on the equivalent circuit diagram shown in FIG. 5 and the electrical variables indicated there.

In FIG. 5, the equivalent circuit diagram of a 2-channel system (two measuring electrodes) with DRL signal injection is shown. In addition, here the parasitic impedance Z_{stray} is taken into account. It is produced by the stray capacitances between the biosignal source and objects of the outside world and also between the measuring system and the outside world. This is particularly considerable in the case of a non-insulated power supply to the measuring system from the grid. Uₚ represents the voltage signal present at the biosignal source with respect to the system ground. The following applies for the coupling impedance of an electrode:

$$\begin{matrix}{\underline{U}}_{out} = {\underline{U}}_{p} \frac{{\underline{Z}}_{in}}{{\underline{Z}}_{in} + {\underline{Z}}_{c}} & ( 3.11 ) \\ {\underline{Z}}_{c} = {\underline{Z}}_{in} ( \frac{{\underline{U}}_{p}}{{\underline{U}}_{out}} - 1 ) & ( 3.12 )\end{matrix}$$

Depending on the angular frequency w of the injection signal, the capacitance and the resistance can be determined from this:

$$\begin{matrix}{C}_{C} = \frac{ ( {\underline{Y}}_{c} )}{ω} = \frac{ ( \frac{1}{{\underline{Z}}_{c}} )}{ω} & ( 3.13 ) \\ \text{?} & ( 3.14 ) \\ {R}_{c} = \frac{1}{ ( {\underline{Y}}_{c} )} = \frac{1}{ ( \frac{1}{{\underline{Z}}_{c}} )} \\ \text{?} \text{indicates text missing or illegible when filed} & ( 3.15 )\end{matrix}$$

The model shows however that the voltage Uₚ is influenced by the impedances Z_{stray}, Z_{drl} and Z_{ci}. It cannot be uniquely determined with the available measuring data.

To be able to determine Uₚ, at least one further injecting electrode is required.

For this purpose, the DRL electrode may be divided into two separate surface areas. As a difference from dividing the measuring electrodes, this does not entail any disadvantage for the signal quality, because in the case of the DRL electrode it is only necessary to maximize the overall capacitance of the two areas. The corresponding equivalent circuit diagram can be seen in FIG. 6; the electrode capacitances and stray capacitances are in this case combined to form the impedance Zₚ. Two injection signals are fed in: Uinj2 with the angular frequency ω2 over both areas and Uᵢₙⱼ₁ with the angular frequency ω₁ only over the first area. The two parts of the electrode respectively contain a shunt Zₛ₁ and Zₛ₂. Measuring the voltage at the shunts allows the complex current intensities I_{S1} and Iₛ₂ for the two angular frequencies ω₁ and ωw₂ to be determined. The shunts should be chosen to be resistive: because the injection signals lie above the ECG bandwidth, the predominantly capacitive coupling impedances Z_{drli}, ωᵢ are smaller with respect to the injection signal than with respect to the ECG signal. The resistive impedances of the shunts remain unchanged however. Consequently, they only produce a small voltage drop in the frequency band of the ECG signal, and consequently do not reduce the effectiveness of the DRL electrode, while the voltage drop in the band of the injection signals becomes greater, and consequently allows a more precise determination of Iₛ. In addition, the model has the input impedances Z,ᵢₙ₁ and Zᵢₙ₂, which model the corresponding parasitic properties of the amplifiers used for the shunt voltage measurement.

It is now intended to show that, with the voltages measured at the shunts, Uₛ₁,ω₂ and U_{s2,ω1} and also U_{s1,ω2} and Uₛ₂, ω₂, the voltage at the patient Uₚ, ω₂ and also the two coupling capacitances Z_{drl1}, ω₂ Z_{drl2}, ω₂ of the DRL electrode can be determined. The method by which the frequency components ω₁ and ω₂ belonging to the respective injection signals Uᵢₙⱼ₁ and Uᵢₙⱼ₂ can be demodulated from the measuring signal has already been described above. In the determination of the voltages and currents with the index ω₁, the voltage source Uᵢₙⱼ₂ is assumed as a short circuit, with the index ω₂-Uᵢₙⱼ₁. To simplify matters, instead of the impedances, the corresponding admittances may be used hereafter. First, the complex current intensities are to be determined by way of the coupling impedances. Kirchhoff's first rule gives:

I_{drl1,ω1}=Y_{s1,ω1}U_{s1,ω1}=(U_{inj,}−U_{s1,ω1})Y_{inj,ωfor i=}1,2 (3.16)

I_{drl2,ω1}=U_{s2,ω1}(Y_{inj,ω1}+Y_{s2,ω1}) (3.17)

I_{drl2,ω2}=Y_{s2,ω2}U_{s2,ω2}−(Uᵢₙⱼ₂−U_{s2,ω2})Y_{in2,ω2} (3.18)

To simplify the further calculation, from here on two assumptions are made:
the two shunts are of such a low resistance in comparison with the coupling impedances that U_{injω}Uₛ applies to both electrodes and frequencies. Uₛ are ignored from now on.
the angular frequencies ω₁ and ω₂ are so close together that Y_{ω1}≈Y_{ω2} applies to all of the admittances. It is assumed from here on that the admittances are frequency-independent.

Kirchhoff's second rule gives:

$$\begin{matrix}\text{?} & ( 3.19 ) \\ \text{?} & ( 3.20 ) \\ \text{?} 0 = {\underline{U}}_{{drl}_{2} , {ω}_{2}} - {\underline{U}}_{{drl}_{1} , {ω}_{2}} = \frac{{\underline{I}}_{{drl}_{2} , {ω}_{2}}}{{\underline{Y}}_{{drl}_{2}}} - \frac{{\underline{I}}_{{drl}_{1} , {ω}_{2}}}{{\underline{Y}}_{{drl}_{1}}} \\ {\underline{Y}}_{{drl}_{2}} = {\underline{Y}}_{{drl}_{1}} \frac{{\underline{I}}_{{drl}_{2} , {ω}_{2}}}{{\underline{I}}_{{drl}_{1} , {ω}_{2}}} \\ 0 = - {\underline{U}}_{{inj}_{1}} + {\underline{U}}_{{drl}_{1} , {ω}_{1}} - {\underline{U}}_{{drl}_{2} , {ω}_{1}} = - {\underline{U}}_{{inj}_{1}} + \frac{{\underline{I}}_{{drl}_{1} , {ω}_{1}}}{{\underline{Y}}_{{drl}_{1}}} - \frac{{\underline{I}}_{{drl}_{2} , {ω}_{1}}}{{\underline{Y}}_{{drl}_{2}}} \\ \text{?} \text{indicates text missing or illegible when filed} & ( 3.21 )\end{matrix}$$

Entering 3.20 into 3.21 and converting produces:

$${\underline{Y}}_{{drl}_{1}} = \frac{1}{{\underline{U}}_{{inj}_{1}}} ( {\underline{I}}_{{drl}_{1} , {ω}_{1}} - \frac{{\underline{I}}_{{drl}_{2} , {ω}_{1}} {\underline{I}}_{{drl}_{1} , {ω}_{2}}}{{\underline{I}}_{{drl}_{2} , {ω}_{2}}} ) \qquad ( 3.22 )$$

The following applies for the voltage Uₚ:

$${\underline{U}}_{p , {ω}_{2}} = {\underline{U}}_{{inj}_{2}} - \frac{{\underline{I}}_{{drl}_{1} , {ω}_{2}}}{{\underline{Y}}_{{drl}_{1}}} = \frac{{\underline{I}}_{{drl}_{2} , {ω}_{2}}}{{\underline{Y}}_{{drl}_{2}}} \qquad ( 3.23 )$$

Consequently, the two coupling impedances of the DRL electrode can be determined from 3.22 and 3.20 and the component of the injection signal at the biosignal source can be determined from 3.23.

The following is a complete listing of all the claims in the application, with an indication of the status of each:

1. A treatment couch for supporting patients in a sitting and/or lying position for a treatment and/or diagnosis, comprising:
a supporting surface comprised of one or more segments and on which the patient is supported during the treatment and/or diagnosis,
multiple capacitive measuring electrodes for the contactless capacitive detection of ECG signals of a patient supported on the supporting surface arranged in at least one segment of the one or more segments of the supporting surface, wherein the multiple capacitive measuring electrodes are on a side of the supporting surface that is near the patient,
at least one electronic signal processing system connected to the measuring electrodes for processing the electrical signals of the measuring electrodes,
at least one injection electrode for feeding injection signals into one or more of the measuring electrodes via the patient supported on the supporting surface,
wherein the electronic signal processing system is designed to determine from the signals received by way of the measuring electrodes, on a basis of signal components that are contained therein and originate from the injection signals, a quality of capacitive coupling of one or more or all of the measuring electrodes to the patient.

2. The treatment couch as claimed in claim 1 wherein one or more or all of the measuring electrodes and/or a first and/or a second injection electrode is/are formed as textile capacitive electrodes which are embedded in a structure near a surface of the side of the supporting surface near the patient.

3. The treatment couch as claimed in claim 1 wherein the electronic signal processing system is arranged on the treatment couch away from the measuring electrodes and/or the injection electrodes.

4. The treatment couch as claimed in claim 1, further comprising: an electrical terminal connector for electrically coupling a treatment monitor to the treatment couch and its at least one electronic signal processing system,
wherein the at least one electronic signal processing system is designed to emit on a basis of the signals of the measuring electrodes ECG signals of the patient in a normalized form by way of the electrical terminal connector.

5. The treatment couch as claimed in claim 1, further comprising at least one electric motor, and wherein at least one segment of the one or more segments is adjustable arbitrarily into different positions by the at least one electric motor.

6. The treatment couch as claimed in claim 1, further comprising at least one electric motor, and wherein the treatment couch is adjustable from a sitting position into a lying position and vice versa by the at least one electric motor.

7. The treatment couch as claimed in claim 1 further comprising multiple fixable rollers for supporting the treatment couch with respect to a floor.

8. The treatment couch as claimed in claim 1, further comprising at least one arm rest, wherein the at least one arm rest is secured to or securable to either a left or right of the supporting.

9. The treatment couch as claimed in claim 1 wherein the at least one electronic signal processing system is designed for determining the quality of the capacitive coupling of a measuring electrode of the multiple capacity measuring electrodes on the basis of amplitude values and phase positions of signal components of the injection signals that are received by way of the measuring electrode.

10. The treatment couch as claimed in claim 1 wherein the at least one electronic signal processing system is designed for determining the heart rate or a variable derived therefrom of the patient supported on the supporting surface.

11. The treatment couch as claimed in claim 1 further comprising at least one acoustic and/or optical signal transmitter, wherein the at least one electronic signal processing system is designed to activate the at least one acoustic and/or optical signal transmitter to issue an alarm signal when there are predetermined signal combinations of detected ECG signals and the quality of the capacitive coupling.

12. The treatment couch as claimed in claim 1 wherein the at least one injection electrode comprises a first injection electrode and a second injection electrode, and wherein the injection signals comprise a first injection signal and a second injection signal, wherein the second injection signal is different from the first injection signal,
wherein the at least one electronic signal processing system is cojnfigured such that the first injection signal is fed into the first injection electrode from the at least one electronic signal processing system and, overlapping in time or at the same time, the second injection signal is fed into the second injection electrode from the at least one electronic signal processing system.

13. The treatment couch as claimed in claim 1 wherein one or more or all of the at least one injection electrodes is/are formed as a capacitive and/or galvanic electrode.
